(19) United States
(12) Patent Application Publication
(10) Pub. No.: US 2023/0270507 A1
(43) Pub. Date: Aug. 31, 2023

(54) Surgical Imaging And Display System, And Related Methods

(71) Applicant: DePuy Synthes Products, Inc., Raynham, MA (US)

(72) Inventors: Julio Duenas, Zuchwil (CH); Mario Mata, Royersford, PA (US); André Furrer, Lüterkofen (CH); Nima Amoi Taleghani, Karlsruhe (DE)

(21) Appl. No.: 18/173,279

(22) Filed: Feb. 23, 2023

Related U.S. Application Data
(60) Provisional application No. 63/314,573, filed on Feb. 28, 2022.

(51) Int. Cl. A61B 34/20; A61B 6/12

(52) U.S. Cl. CPC A61B 34/20; A61B 6/12; A61B 2090/376

(57) ABSTRACT

A medical imaging system includes a robotic arm carrying a fluoroscopic imager for generating fluoroscopic image data of anatomy along a beam axis. The arm can adjust a relative position between the fluoroscopic imager and the anatomy. A video imager generates video image data of the anatomy along a sightline axis. A marker is positionable relative to the anatomy and defines a feature for capture in the fluoroscopic and video image data. A processor is configured to execute instructions upon the fluoroscopic and video image data and: (a) register a reference position of the feature relative to the anatomy in the fluoroscopic and video image data; and (b) generate an augmented image stream showing the fluoroscopic or video image data overlaid onto the other such that the reference positions are co-registered. The system includes a display configured to present the augmented image stream of the anatomy substantially in real time.

### TECHNICAL FIELD

The present invention relates to systems that can be used in conjunction with medical imaging.

### BACKGROUND

A C-arm, or a mobile intensifier device, is one example of a medical imaging device that is based on X-ray technology. The name C-arm is derived from the C-shaped arm used to connect an X-ray source and an X-ray detector with one another. Various medical imaging devices, such as a C-arm device, can perform fluoroscopy, which is a type of medical imaging that shows a continuous X-ray image on a monitor. During a fluoroscopy procedure, the X-ray source or transmitter emits X-rays that penetrate a patient's body. The X-ray detector or image intensifier converts the X-rays that pass through the body into a visible image that is displayed on a monitor of the medical imaging device. Medical professionals can use such imaging devices, for example, to assess bone fractures, guide surgical procedures, or verify results of surgical repairs. Because medical imaging devices such as a C-arm device can display high-resolution X-ray images in real time, a physician can monitor progress at any time during an operation, and thus can take appropriate actions based on the displayed images.

In various embodiments described herein, images provided by imaging devices are transmitted in real-time to a display that can be mounted to an apparatus of the surgical system, such as a workstation near the operating table or directly on a surgical instrument, such that fluoroscopic imaging provided by the imaging device can be viewed by a medical professional as the medical professional operates and views a working end of the surgical instrument. The display can receive the images in real-time, such that the images are displayed by the display at the same time that the images are generated by the imaging device.

Monitoring the images, however, is often challenging during certain procedures, for instance during procedures in which attention must be paid to the patient's anatomy as well as the display of the medical imaging device. For example, aligning a drill bit to a distal locking hole can be difficult if a medical professional is required to maneuver the drill while viewing the display of the medical imaging device that is outside of the field of view of the medical procedure.

### SUMMARY

According to an embodiment of the present disclosure, a medical imaging system includes a robotic arm carrying a fluoroscopic imaging device having an x-ray transmitter, wherein the fluoroscopic imaging device is configured to generate fluoroscopic image data of an anatomical structure along a beam axis. The robotic arm is manipulatable for adjusting a relative position between the fluoroscopic imaging device and the anatomical structure. The system also includes a video imaging device configured to generate video image data of the anatomical structure along a camera sightline axis, and a marker that is can be positioned with respect to the anatomical structure. The marker defines at least one reference feature configured to be captured in the fluoroscopic image data and the video image data. A processor is in communication with the fluoroscopic imaging device and the video imaging device and also with a memory having instructions stored therein. The processor is configured to execute the instructions upon the fluoroscopic image data and the video image data and responsively: (a) register a reference position of the at least one reference feature relative to the anatomical structure in the fluoroscopic image data and the video image data; and (b) generate an augmented image stream that shows one of the fluoroscopic image data and the video image data overlaid onto the other of the fluoroscopic image data and the video image data such that the reference positions are co-registered. The system also includes a display in communication with the processor, wherein the display is configured to present the augmented image stream of the anatomical structure substantially in real time.

According to another embodiment of the present disclosure, a method includes steps of generating a fluoroscopic stream of images of an anatomical structure, generating a video stream of images of the anatomical structure, co-registering the fluoroscopic stream of images with the video stream of images, and depicting, on a display, an augmented image stream that includes the co-registered fluoroscopic stream of images overlaid over the co-registered video stream of images.

According to yet another embodiment of the present disclosure, a surgical system includes a robotic arm carrying a fluoroscopic imaging device having an x-ray transmitter, wherein the fluoroscopic imaging device is configured to generate a first stream of fluoroscopic images of an anatomical structure along a first beam axis at a first orientation relative to the anatomical structure. The fluoroscopic imaging device is also configured to generate a second stream of fluoroscopic images of the anatomical structure along a second beam axis at a second orientation relative to the anatomical structure, wherein the second beam axis intersects the first beam axis and is substantially perpendicular to the first beam axis. The robotic arm is manipulatable for adjusting a relative position between the fluoroscopic imaging device and the anatomical structure. The system includes a processor in communication with the fluoroscopic imaging device and the robotic arm. The processor is further in communication with a memory having instructions stored therein, such that the processor is configured to execute the instructions upon the first and second streams of fluoroscopic images and responsively: (a) identify at least one anchor hole in an implant that resides within the anatomical structure; (b) reposition the fluoroscopic imaging device so that the first beam axis extends orthogonal to the at least one anchor hole; and (c) plot, in the second stream of fluoroscopic images, a reference axis that extends centrally through the at least one hole. The system also includes a display in communication with the processor, wherein the display is configured to depict an augmented version of the second stream of fluoroscopic images that shows the reference axis overlaying the anatomical structure.

According to an additional embodiment of the present disclosure, a method includes steps of generating a first fluoroscopic stream of images along a first beam axis, such that the first fluoroscopic stream shows an implant residing in an anatomical structure. A second fluoroscopic stream of images of the anatomical structure is generated along a second beam axis that intersects the first beam axis at an angle. The method includes processing the first and second fluoroscopic streams of images with a processor in communication with memory. This processing step comprises (a) identifying a reference feature of the implant, (b) calculating a pixel ratio of the reference feature in pixels per unit length, (c) adjusting an orientation of the first beam axis so that it extends orthogonal to the reference feature, (d) generating a reference axis extending centrally through the reference feature such that the reference axis is parallel with the first beam axis, and (e) depicting the second image stream on a display, such that the reference axis is depicted in the second image stream overlaying the anatomical structure.

The foregoing summarizes only a few aspects of the present disclosure and is not intended to be reflective of the full scope of the present disclosure. Additional features and advantages of the disclosure are set forth in the following description, may be apparent from the description, or may be learned by practicing the invention. Moreover, both the foregoing summary and following detailed description are exemplary and explanatory and are intended to provide further explanation of the disclosure.

### BRIEF DESCRIPTION OF THE DRAWINGS

The foregoing summary, as well as the following detailed description of example embodiments of the present disclosure, will be better understood when read in conjunction with the appended drawings. For the purposes of illustrating the example embodiments of the present disclosure, references to the drawings are made. It should be understood, however, that the application is not limited to the precise arrangements and instrumentalities shown. In the drawings:

FIG. 1 depicts an example imaging system in accordance with an example embodiment, wherein the example imaging system includes a fluoroscopic imaging device and a video imaging device for generating augmented reality (AR) medical imagery;

FIGS. 2A-2B are a block diagrams of example computing devices for use in the imaging system shown in FIG. 1;

FIG. 3A is a perspective view of a robotic arm carrying the imaging devices of the imaging system illustrated in FIG. 1, wherein an anatomical structure is positioned within a field of view of the imaging devices;

FIG. 3B shows an example X-ray image generated by the fluoroscopic imaging device of FIG. 3A;

FIG. 3C shows an example video image generated by the video imaging device of FIG. 3A;

FIG. 3D shows an example AR image produced by the imaging system, wherein the AR image includes the fluoroscopic image overlaid on the video image in an anatomically matching configuration;

FIG. 4A is a perspective view of a robotic arm according to another example embodiment of an imaging system for generating augmented reality (AR) medical imagery, wherein the robotic arm carries the fluoroscopic imaging device, and the video imaging device is located on a surgical instrument configured to operate upon the anatomical structure within the field of view of the fluoroscopic imaging device;

FIG. 4B shows an example X-ray image generated by the fluoroscopic imaging device of FIG. 4A;

FIG. 4C shows an example video image generated by the video imaging device of FIG. 4A;

FIG. 4D shows an example AR image produced by the imaging system, wherein the AR image includes the X-ray image overlaid on the video image in an anatomically matching configuration;

FIG. 4E is a block diagram of example computing devices for use with the example imaging system shown in FIG. 4A;

FIG. 5 is a process diagram illustrating an example process employed by the imaging system to generate the AR images illustrated in FIGS. 3D and 4D;

FIG. 6 is a flowchart showing an example method of processing video images generated by the video imaging devices illustrated in FIGS. 3A and 4A;

FIG. 7 is a flowchart showing an example method of processing X-ray images generated by the fluoroscopic imaging devices illustrated in FIGS. 3A and 4A;

FIG. 8 depicts an example imaging system in accordance with another example embodiment, wherein the example imaging system includes first and second fluoroscopic imaging devices that view the same anatomical region from angularly offset positions;

FIG. 9 is a flowchart showing an example process for using the imaging system illustrated in FIG. 8 to calculate the required orientation and length of anchors in three-dimensional (3D) space for anchoring an implant to the anatomical structure;

FIGS. 10A-10D show example fluoroscopic images produced during the process illustrated in FIG. 9; and

FIG. 11 is a flowchart showing an example method of processing fluoroscopic images generated by the fluoroscopic imaging devices illustrated in FIG. 8.

### DETAILED DESCRIPTION

In various embodiments described herein, images provided by imaging devices are transmitted in real-time to a display that can be mounted to an apparatus of the surgical system, such as a workstation near the operating table or directly on a surgical instrument, such that fluoroscopic imaging provided by the imaging device can be viewed by a medical professional as the medical professional operates and views a working end of the surgical instrument. The display can receive the images in real-time, such that the images are displayed by the display at the same time that the images are generated by the imaging device.

However, fluoroscopic images alone can omit critical information about patient anatomy and/or surgical components at a surgical treatment site, such as the location and orientation of target features of an implant with respect to the surgeon, according to one non-limiting example, and/or the precise spatial relationships between various portions of the anatomy, according to another non-limiting example, and/or a combination of the foregoing examples of critical information. Accordingly, an enhanced surgical imaging system that can generate and display augmented fluoroscopic images containing critical supplemental information would provide numerous benefits to the patient, for example, by allowing surgeons to complete surgical procedures with greater accuracy and more efficiently, thereby reducing the amount of X-ray exposure imposed on the patient (and also on the surgeon and staff).

The following disclosure describes various embodiments of surgical imaging systems that employ a fluoroscopic imaging device with an additional imaging device and uses the image data from both imaging devices to generate and display augmented fluoroscopic images that presents information obtained from both imaging devices. These augmented fluoroscopic images provide the surgeon with critical supplemental information necessary to complete various surgical procedures with greater accuracy and efficiency. By way of non-limiting examples, the various embodiments described below are expected to reduce the time necessary to complete an intramedullary (IM) nailing procedure, particularly by providing faster and more accurate techniques for determining necessary anchor length for distal locking, and also by providing simpler techniques for targeting distal locking holes of the IM nail.

In one example, the display presents an augmented image stream that includes fluoroscopic images of the treatment site paired with and superimposed onto video images of the treatment site in a continuous “augmented reality” stream, allowing the surgeon to more rapidly identify the location of distal locking holes relative to a tip of an associated surgical drill. In this example, the video camera can be mounted to the C-arm or the instrument (e.g., a surgical drill). In another example, the display presents an augmented image stream generated from two separate but intersecting fluoroscopic image streams, which allows a control system to identify target features of an implant residing in an anatomical structure and also to calculate the required orientation and length of anchors in three-dimensional (3D) space for insertion through anchor holes of the implant for anchorage to the anatomical structure. It should be appreciated that the foregoing examples are provided as non-limiting examples of the surgical imaging systems of the present disclosure.

As an initial matter, because fluoroscopy is a type of medical imaging that shows a continuous X-ray image on a monitor, the terms fluoroscopic data, fluoroscopic image, video data, and X-ray image may be used interchangeably herein, without limitation, unless otherwise specified. Thus, an X-ray image may refer to an image generated during a fluoroscopic procedure in which an X-ray beam is passed through the anatomy of a patient. Further, it will be understood that fluoroscopic data can include an X-ray image, video data, or computer-generated visual representations. Thus, fluoroscopic data can include still images or moving images.

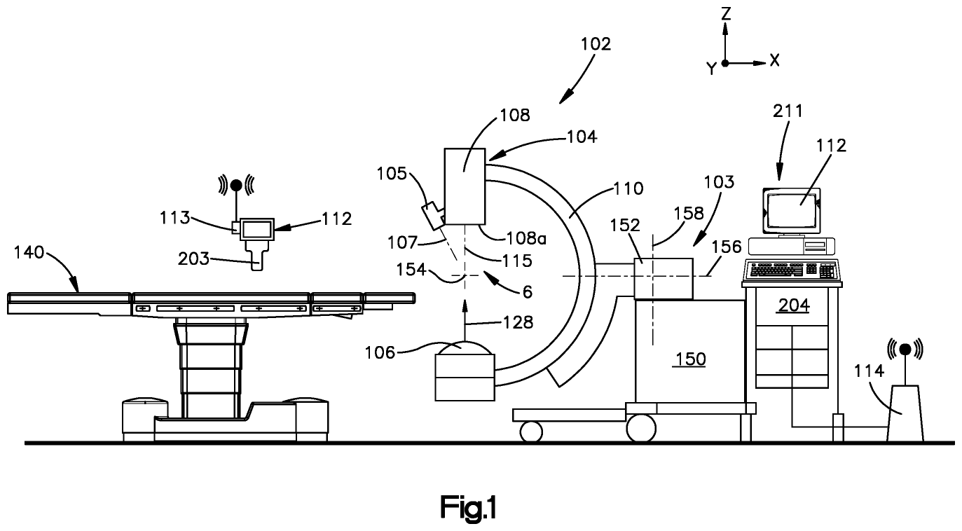

Referring to FIG. 1, an example surgical imaging system 102 is shown for generating and displaying augmented imagery, such as a stream of augmented images, showing an anatomical structure 4 during a medical imaging procedure, such as a surgical imaging procedure. In particular, the surgical imaging system 102 is configured such that the augmented imagery includes a first stream of images, such as a stream of fluoroscopic images, of a surgical treatment site matched with and overlapped with a second stream of images, such as a stream of video images, of the treatment site. Accordingly, the surgical imaging system 102 of the present embodiment can be referred to as an “augmented reality” (AR) surgical imaging system 102, and the augmented imagery can be referred to as augmented reality (AR) imagery. The AR surgical imaging system 102 can include an imaging station 103 that includes a positioning mechanism, such as a robotic arm 110, that carries a first imaging device 104, such as a fluoroscopic imaging device 104. The fluoroscopic imaging device 104 is configured to generate fluoroscopic image data, such as X-ray images, including a continuous stream of X-ray images, of the anatomical structure 4.

The robotic arm 110 can be a C-arm or similar type device, by way of non-limiting example. The fluoroscopic imaging device 104 can include an X-ray generator or transmitter 106 configured to transmit X-rays through a body (e.g., bone) along a central beam axis 115 (also referred to herein as the “beam axis” 115). The fluoroscopic imaging device 104 can also include an X-ray detector or receiver 108 configured to receive the X-rays from the X-ray transmitter 106. Thus, the fluoroscopic imaging device 104 can define a direction of X-ray travel 128 from the X-ray transmitter 106 to the X-ray receiver 108. The direction of X-ray travel 128 is parallel and/or colinear with the beam axis 115. The X-ray transmitter 106 can define a flat surface 106a that faces the X-ray receiver 108. The area between the X-ray transmitter 106 and detector 108 can be referred to as the “imaging zone” 6 of the fluoroscopic imaging device 104. The robotic arm 110 can physically connect the X-ray transmitter 106 with the X-ray receiver 108.

The fluoroscopic imaging device 104 is configured to be in communication with an AR display 112 that is configured to display the AR imagery, which is generated in part from the fluoroscopic image data, as described in more detail below.

The AR surgical imaging system 102 can include a support apparatus 140, such as a table 140, for supporting a patient during the medical imaging procedure so that the anatomical region of interest (ROI) (e.g., the anatomical structure 4 at the surgical treatment site) is positioned between the X-ray transmitter 106 and the X-ray detector 108 and is thereby intersected by the X-rays.

The robotic arm 110 is preferably manipulatable with respect to one or more axes of movement for adjusting a relative position between the fluoroscopic imaging device 104 and the anatomical structure 4. For example, the imaging station 103 can include a base 150 that supports the robotic arm 110. The robotic arm 110 can include an actuation mechanism 152 that adjusts the position of the robotic arm 110 with respect to the base 150, such as along one or more axes of movement. For example, the actuation mechanism 152 can be configured to pivot the robotic arm 110 about a central pivot axis 154, which can extend centrally between the X-ray transmitter and detector 106, 108 along a lateral direction Y and intersect the beam axis 115 perpendicularly at a central reference point 155. Additionally or alternatively, the actuation mechanism 152 can translate the robotic arm 110 forward and rearward along a longitudinal axis 156 oriented along a longitudinal direction X. The actuation mechanism 152 can additionally or alternatively raise and lower the robotic arm 110 along a vertical axis 158 oriented along a vertical direction Z. The longitudinal, lateral, and vertical directions X, Y, Z can be substantially perpendicular to each other. The actuation mechanism 152 can optionally further pivot the robotic arm 110 about one or both of the longitudinal and vertical axes 156, 158. In manner described above, the robotic arm 110 can be provided with multi-axis adjustability for obtaining images of the anatomical structure 4 at precise locations and orientations. By way of a non-limiting example, the table 140 (and the anatomical structure 4 thereon) can be brought into the imaging zone 6, and the actuation mechanism 152 can be employed to manipulate the relative position between the robotic arm 110 and the anatomical structure 4 such that the central reference point 155 is centered at a location of interest of the anatomical structure 4. From this centered position, the robotic arm 110 can be rotated as needed, such as about axis 154, to obtain fluoroscopic image data at multiple angles and orientations with the location of interest (i.e., at the central reference point 155) centered in the images.

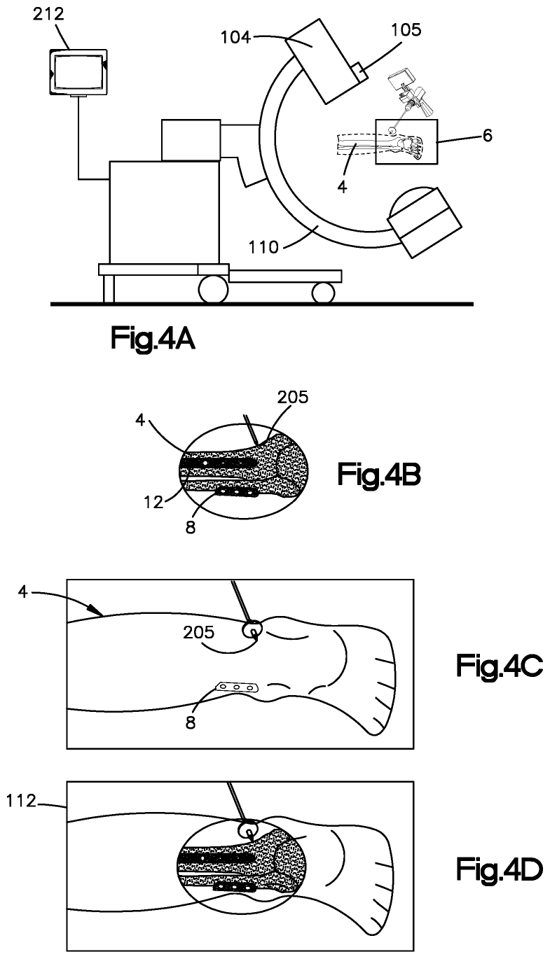

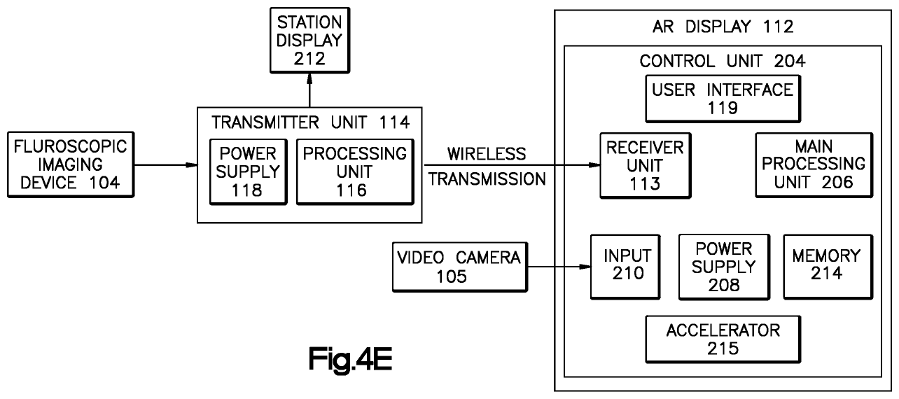

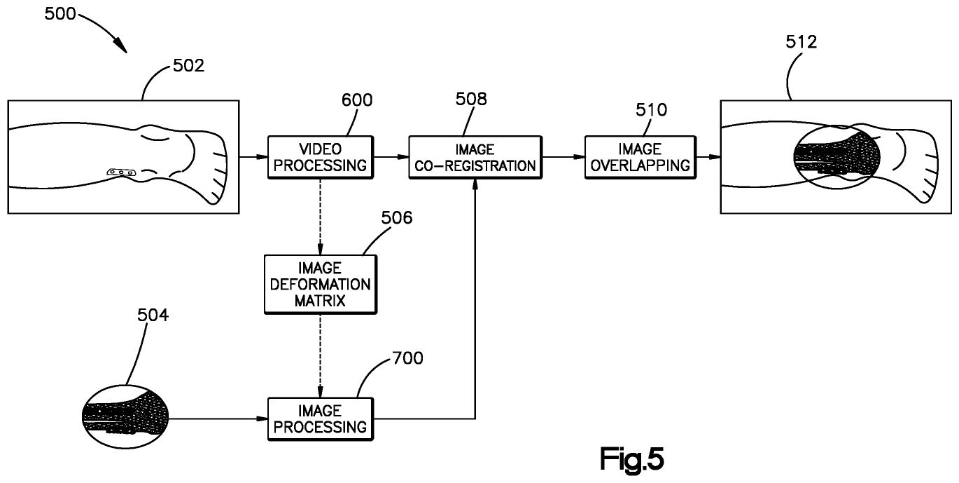

The AR surgical imaging system 102 includes a second imaging device 105, which in the present embodiment is preferably a video camera 105. The first and second imaging devices 104, 105 can define an imaging array. As shown, the camera 105 can be mounted to the robotic arm 110 in a manner to capture video images of a field of view of the fluoroscopic imaging device 104. In other embodiments (see FIG. 4A), the camera 105 can be remote from the robotic arm 110, as will be described in more detail below. The camera 105 is configured to generate second image data (also referred to herein as “camera image data”), such as images, including a continuous stream of images (i.e., a video stream), along a camera sightline axis 107. The camera image data is used in combination with the fluoroscopic image data to generate the AR imagery for displaying on the AR display 112. The camera 105 can be oriented such that camera sightline axis 107 is substantially parallel with the beam axis 115. In other embodiments, the camera sightline axis 107 can be angularly offset from the beam axis 115.

The AR surgical imaging system 102 can include one or more surgical instruments 203 for guided use with the AR display 112. In the present embodiment, the one or more surgical instruments 203 include a power drill 203 for targeting locking holes of an implant 12 (see FIG. 4B). It should be appreciated that other types of surgical instruments can be employed with the AR surgical imaging system 102. In the illustrated embodiment, the AR display 112 is mountable to the surgical instrument 203. In other embodiments, the AR display 112 can be mountable to the imaging station 103, to the table 140, or at another location within the AR surgical imaging system 102. In further embodiments, the AR display 112 can be a mobile type of AR display 112, such as a tablet, smart phone, headset visor, or the like, that can be carried and/or worn by a physician.

The AR surgical imaging system 102 includes an electronic control unit (ECU) 204 (also referred to herein as a “control unit”) that is configured to generate the AR imagery, such as a continuous stream of AR images that includes the fluoroscopic image data overlapped with the second image data (i.e., the camera image data). In particular, the control unit 204 is configured to overlap the fluoroscopic and camera image data in an anatomically matching configuration. It should be appreciated that the control unit 204 can include, or be incorporated within, any suitable computing device configurable to generate the AR imagery. Non-limiting examples of such computing devices include a station-type computer, such as a desktop computer, a computer tower, or the like, or a portable computing device, such as a laptop, tablet, smart phone, or the like. In the illustrated embodiment, the control unit 204 is incorporated into computer station 211 that is integrated into or with the fluoroscopic imaging device 104. In other embodiments, the control unit 204 can be incorporated into a computer station 211 that can be mobile with respect to the fluoroscopic imaging device 104 with a wired or wireless electronic communication therewith. In further embodiments, the control unit 204 can be coupled to or internal to the surgical instrument 203, as described in more detail below.

The AR surgical imaging system 102 can also include a transmitter unit 114, which can be configured to communicate image data between the imaging station 103 and the AR display 112. In the illustrated embodiment, the transmitter unit 114 is electronically coupled (e.g., wired) to the control unit 204, which receives the fluoroscopy image data from the fluoroscopic imaging device 104 and also receives the camera image data from the video camera 105 and overlaps the fluoroscopic and camera image data to generate the AR imagery. The transmitter unit 114 then wirelessly transmits the AR imagery to a receiver unit 113 that is integrated with or connectable to the AR display 112. In such embodiments, the AR imagery is generated at the computer station 211 and subsequently transmitted, via the transmitter and receiver units 114, 113, to the AR display 112, which then displays the transmitted AR imagery to a physician. The transmitter unit 114 can be integrated with the control unit 204 or can be a separate unit electrically coupled thereto. The transmitter unit 114 can be any suitable computing device configured to receive and send images, such as the AR imagery. Non-limiting examples of such computing devices include those found in a portable computing device, such as in a laptop, tablet, smart phone, or the like.

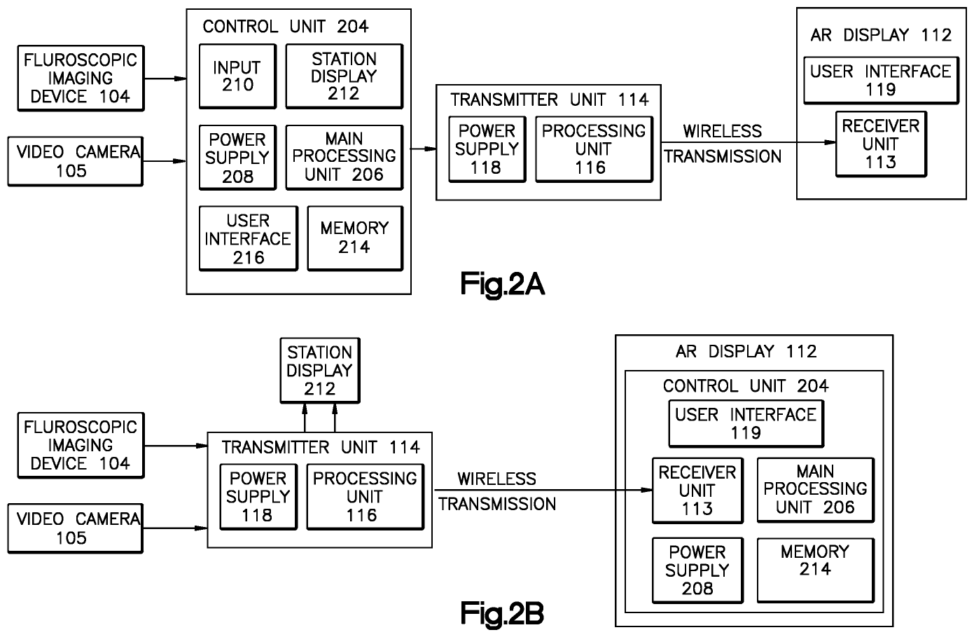

Referring now to FIG. 2A, in one example embodiment, the control unit 204 includes a main processing unit or “processor” 206, a power supply 208, an input portion 210, and a memory portion 214 (also referred to herein as “memory” 214). In particular, the main processor 206 is configured to receive the fluoroscopic and camera image data from the input portion 210 and execute machine-readable instructions (e.g., image processing instructions and augmentation instructions) to overlap the fluoroscopic image data and the camera image data and thereby generate the AR imagery. The machine-readable instructions can also include other instructions, such as for operating the imaging station 103, such as for positioning the robotic arm 110 to locate the ROI within the imaging zone 6.

The control unit 204 can include a station display 212 and a user interface 216 having controls 219 for receiving user inputs for controlling one or more operations of the control unit 204. It should be understood that the station display 212 is separate from the AR display 112 described above. The main processor 206, input portion 210, station display 212, memory 214, and user interface 216 are preferably in communication with each other or at least connectable to provide communication therebetween. It should be appreciated that any of the above components may be distributed across one or more separate devices and/or locations. The station display 212 can be mounted at the computer station 211 and can be configured to display the fluoroscopic image data from the fluoroscopic imaging device 104 and/or the camera image data from the video camera 105. In this manner, the station display 212 can be employed to ensure that the ROI is positioned within the imaging zone 6. In some embodiments, the station display 212 can provide split-screen functionality to separately display both the fluoroscopic image data and the camera image data in real time. In various embodiments, the input portion 210 of the control unit 204 can include one or more receivers. The input portion 210 is capable of receiving information in real time, such as the fluoroscopic image data and the camera image data, and delivering the information to the main processor 206. It should be appreciated that receiver functionality of the input portion 210 may also be provided by one or more devices external to the control unit 204.

The memory 214 can store instructions therein that, upon execution by the main processor 206, cause the control unit 204 to perform operations, such as the augmentation operations described herein. Depending upon the exact configuration and type of processor, the memory 214 can be volatile (such as some types of RAM), non-volatile (such as ROM, flash memory, etc.), or a combination thereof. The control unit 204 can include additional storage (e.g., removable storage and/or non-removable storage) including, but not limited to, tape, flash memory, smart cards, CD-ROM, digital versatile disks (DVD) or other optical storage, magnetic cassettes, magnetic tape, magnetic disk storage or other magnetic storage devices, universal serial bus (USB) compatible memory, or any other medium which can be used to store information and which can be accessed by the control unit 204.

The user interface 216 is configured to allow a user to communicate with and affect operation of the control unit 204. The user interface 216 can include inputs or controls 219 that provide the ability to control the control unit 204, via, for example, buttons, soft keys, a mouse, voice actuated controls, a touch screen, a stylus, movement of the control unit 204, visual cues (e.g., moving a hand in front of a camera), or the like. The user interface 216 can provide outputs, including visual information (e.g., via the station display 212), audio information (e.g., via speaker), mechanically (e.g., via a vibrating mechanism), or a combination thereof. In various configurations, the user interface 216 can include the station display 212, a touch screen, a keyboard, a mouse, an accelerometer, a motion detector, a speaker, a microphone, a camera, a tilt sensor, or any combination thereof.

The transmitter unit 114 can include an independent power supply 118 and can also include an independent, secondary processing unit 116 for adjusting the wireless transmission signal (e.g., amplitude, frequency, phase) as needed before or during wireless transmission to the receiver unit 113.

The receiver unit 113 can include any suitable computing device configured to receive wireless transmission of images, particularly the AR imagery. Non-limiting examples of such computing devices include those found in portable computing devices, such as a laptop, tablet, smart phone, and the like. It should be appreciated that the receiver unit 113 can also include an independent power supply and can also include an independent, secondary processing unit for adjusting the AR imagery (e.g., brightness, contrast, scale) as needed to enhance the visual perception displayed on the AR display 112. The AR display 112 also includes a user interface 119 in communication with controls for receiving user inputs for controlling one or more operations of the AR display 112, such as ON/OFF functionality and operations to be executed by the secondary processing unit, such as image adjustment (e.g., brightness, contrast, scale) and the like. The user interface 119 of the AR display 112 can include a graphical user interface (GUI) and/or other types of user interfaces. The user interface 119 can be operated by various types of controls and/or inputs, such as touch-screen controls, buttons, dials, toggle switches, or combinations thereof.

Referring now to FIG. 2B, in another example embodiment, the control unit 204 is integrated with or coupled to the AR display 112, whereby the AR imagery is both generated and displayed at the AR display 112. In this example, the transmitter unit 114 receives the fluoroscopic and camera image data from the fluoroscopic imaging device 104 and the video camera 105, respectively, and wirelessly transmits the fluoroscopic and camera image data to the control unit 204. As shown, the transmitter unit 114 can also communicate the fluoroscopic and camera image data to a station display 212, which can be configured similar to the station display 212 described above. In this embodiment, the control unit 204 includes a receiver unit 113, which can be configured similarly to the receiver unit 113 described above. In the present embodiment, the receiver unit 113 is configured to receive the wireless fluoroscopic and camera image data from the transmitter unit 114 and convey the data to the main processor 206 for image processing and generating the AR imagery, which is displayed on the AR display. In the present embodiment, the memory 214 is integrated with the AR display 112. It should be appreciated that, in the present embodiment, the main processor 206 can also communicate with the user interface 119 of the AR display 112 for controlling other operations of the AR display 112 (e.g., ON/OFF functionality, image adjustment, and the like). Because the receiver unit 113 and the main processor 206 in the present embodiment are both part of the control unit 204, the receiver unit 113 optionally need not have an independent, secondary processor.

It should be appreciated that the block diagram depictions of the transmitter units 114 and the control units 204 shown in FIGS. 2A-2B are provided as examples and are not intended to limit the AR surgical imaging system 102 of the present disclosure to specific implementations and/or configurations. It should also be appreciated that the transmitter unit 114 and/or the control unit 204 can operate and/or can be configured as more fully described in U.S. Pat. No. 11,166,766, issued Nov. 9, 2021, and entitled “Surgical Instrument Mounted Display System” (hereinafter “the '766 Reference”) the entire disclosure of which is incorporated herein by this reference.

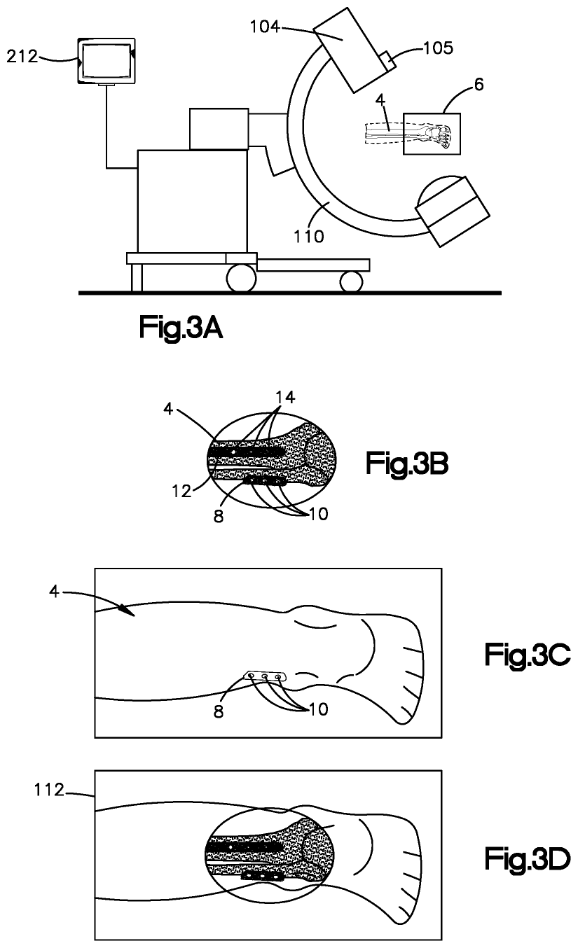

Various techniques can be employed to achieve the anatomical matching configuration of the AR images. Non-limiting examples of such techniques will now be described with reference to FIGS. 3A-7.

Referring now to FIGS. 3A-3D, an object, such as a reference marker or “marker” 8 having at least one reference feature 10, can be positioned with respect to the anatomical structure 4 so that the reference feature(s) 10 is within a field of view of one or both of the fluoroscopic imaging device 104 and the camera 105. In this manner, the reference feature(s) 10 can be captured in at least one of the fluoroscopic image data and the camera image data. In the present embodiment, the reference feature(s) 10 is preferably positioned within the ROI, which is positioned within the imaging zone 6 of the fluoroscopic imaging device 104 so that it can be captured in both of the fluoroscopic image data and the camera image data. Accordingly, in this embodiment the marker 8 is preferably radiopaque and is positioned at an ex vivo location adjacent the anatomical structure 4 within the ROI.

As shown in FIGS. 3B and 3C, the reference feature(s) 10 can be defined by one or more holes, preferably through-holes, extending orthogonally between opposed planar surfaces of the marker 8. In this manner, each reference feature 10 defines a specific shape (“reference shape”) in a reference plane. The marker 8 preferably has opposite ends that are shaped differently from each other so that the orientation of the marker 8 in the fluoroscopic image data and camera image data is more readily discernable. With the marker 8 in place and the ROI positioned within the imaging zone 6, the fluoroscopic imaging device 104 obtains fluoroscopic image data in which the marker 8 is discernible and the camera 105 obtains camera image data in which the marker 8 is also discernible. The control unit processes the fluoroscopic and camera image data to identify the reference feature(s) 10 (e.g., hole(s)) of the marker 8 therein and uses the reference feature(s) 10 to generate the AR images having the fluoroscopic and camera images overlapped in the anatomically matching configuration, as shown in FIG. 3D. It should be appreciated that the marker 8 shown in FIGS. 3B-3D represents a non-limiting example of the shape and type of marker that can be employed with the AR surgical imaging system 102. Various other marker shapes, types, and reference feature geometries, are within the scope of the embodiments herein. It should be appreciated that such other marker shapes, types, and reference feature geometries are preferably radiopaque so as to be visible in X-ray imagery.

Referring now to FIGS. 4A-4D, in other embodiments, the video camera 105 can be located on a surgical instrument 203 configured to operate on the anatomical structure 4. As shown in FIG. 4E, in such embodiments, the control unit 204 is preferably integrated with or coupled to the AR display 112. For example, the control unit 204 can be internally located in the surgical tool 203 and can have a wired or wireless connection with the AR display 112. The transmitter unit 114 transmits the fluoroscopic image data to the receiver unit 213 of the AR display 112. The control unit 204 can include an input 210, which is configured similar to that described above, and that receives the camera image data from the video camera 105. The receiver unit 113 and the input 210 deliver the fluoroscopic and camera image data, respectively, to the main processor 206 for image processing and generating the AR imagery.

As described above, the marker 8 is positioned within the ROI, which is positioned within the imaging zone 6 of the fluoroscopic imaging device 104 so that the marker 8 is captured in the fluoroscopic image data. In the present embodiment, when the surgical instrument 203 is directed toward the ROI, the marker 8 can also be captured in the camera image data. The fluoroscopic imaging device 104 obtains fluoroscopic image data in which the marker 8 is discernible (FIG. 4B) and the camera 105 obtains camera image data in which the marker 8 is also discernible (FIG. 4C). The transmitter unit 114 processes the fluoroscopic data, identifies and employs the reference feature(s) 10, and transmits the resulting fluoroscopic image data to the receiver unit 113 (FIG. 4E). The receiver unit 113 delivers the transmitted fluoroscopic image data to the main processor 206, wherein the fluoroscopic image data and the camera image data will be use to generate the AR imagery, as shown in FIG. 4D. The control unit 204 can optionally include an accelerometer 215 (FIG. 4E), which can be configured to generate accelerometer information that can allow the control unit 204 to calculate an orientation of the surgical instrument 203 with respect to the fluoroscopic imaging device 104, as more fully described in the '766 Reference.

Referring now to FIG. 5, an example augmentation algorithm or process 500 for generating the AR imagery can include steps 502, 504, 600, 506, 700, 508, 510, and 512. It should be appreciated that, although the example augmentation process 500 described below utilizes the processed video images as “base” or “reference” images and the processed X-ray images as the “source” or “slave” images during the overlapping or superimposition process, alternative processes can utilize the X-ray images as the reference images and the video images as the source images. Step 502 includes obtaining the camera image data using the camera 105 and transmitting the camera image data to the control unit 204. Step 504 includes obtaining the fluoroscopic image data using the fluoroscopic imaging device 104 and transmitting the fluoroscopic image data to the control unit 204, such as in DICOM format, by way of a non-limiting example. It should be appreciated that steps 502, 504 can be performed in the manners described above with reference to FIGS. 3A-3C and 4A-4C. It should also be appreciated that at least steps 600, 506, 700, 508, and 510 are performed by the control unit 204, particularly by the main processor 206 executing program instructions stored in the memory 214. As described above, the control unit 204 can be located at the computer station 211 or at the AR display 112, depending on the particular embodiment. Step 600 includes processing the camera image data showing the marker 8 to generate an image deformation matrix 506 (also referred to as a “transformation matrix”). Step 700 includes processing the fluoroscopic image data showing the marker 8 for comparison with the image deformation matrix 506. Step 508 includes co-registration of the processed fluoroscopic image data with the processed camera image data. Step 510 includes overlapping (superimposing) the co-registered fluoroscopic image data and camera image data into an AR image stream. Step 512 includes displaying the AR image stream on the AR display 112.

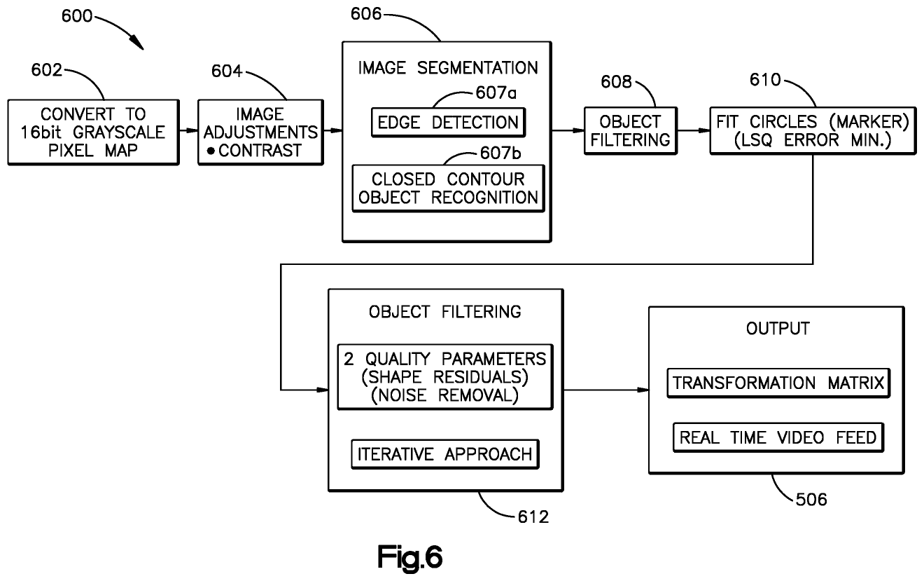

Referring now to FIG. 6, further details of step 600 (processing the camera image data) will now be described. Step 600 can include sub-steps 602, 604, 606, 608, 610, and 612, which can each be referred to as a step. Step 602, which is optional, includes mapping each image of the video stream, such as by converting each image to a 16-bit grayscale pixel map, which can have various pixel matrix configurations. By way of non-limiting examples, the pixel matrix configuration of the pixel map can be from 8×32 to 16×32 and preferably from 8×64 to 16×64, depending on the C-arm employed. As another non-limiting example, the pixel map can be an 8-bit pixel map. Step 604 includes adjusting each image for subsequent processing, such as by adjusting the contrast of each image as needed. Step 606 includes performing image segmentation on each image, which can include a sub-step 607a of performing edge detection on each image, and can include another sub-step 607b of performing object recognition within each image, such as by comparing image data in each image to a library of reference images stored in the computer memory. Step 608 includes object filtering on the segmented images. The object filtering in step 608 can be performed according to one or more various quality parameters. Step 610 includes fitting a shape with respect to each reference feature 10 (e.g., hole) of the marker 8. For example, in the illustrated embodiments, this step 610 includes fitting reference circles with the holes of the marker 8. For example, the shape(s) can be fitted using least squares approximation (LSQ) techniques, such as an LSQ minimum error approximation. Step 612 includes further object filtering the images processed according to step 610 (e.g., having a shape fitted to each reference feature 10). The object filtering in step 612 can be performed according to two (2) or more quality parameters, which can include shape residuals and noise removal, by way of non-limiting examples. Step 612 can also be performed according to an iterative approach, in which the outcome of one or more of the filtering parameters is reapplied to each image, such as until a predetermined filtering standard or threshold is achieved for each image. At step 506, the video images processed in step 612, particularly the marker 8 and its reference feature(s) 10 therein, are each transform-processed to calculate an image deformation matrix for each image, which are then streamed in sequence in a processed real-time video stream.

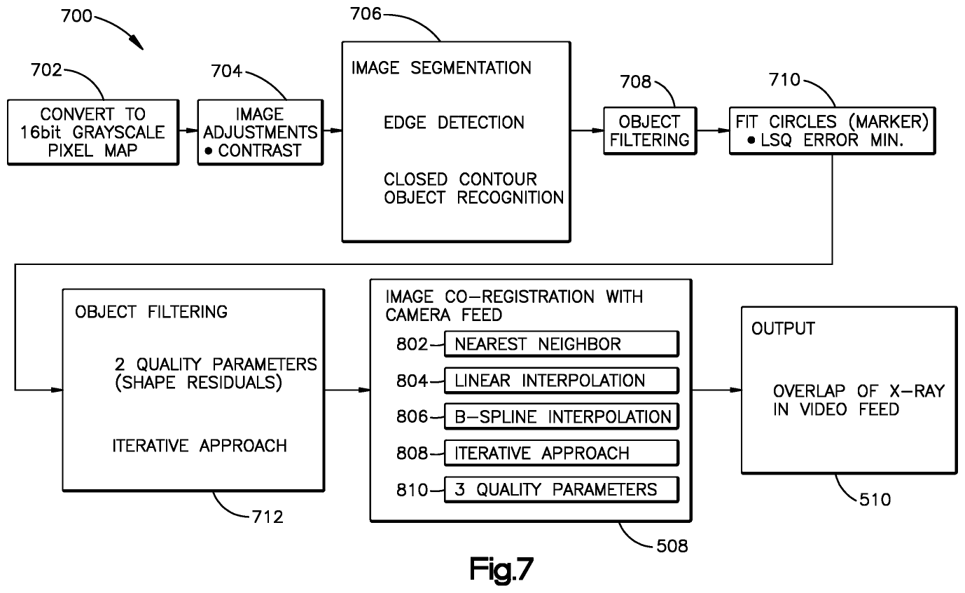

Referring now to FIG. 7, further details of the fluoroscopic image data processing 700 step will now be described. Step 700 can include sub-steps 702, 704, 706, 708, 710, and 712, which can each be referred to as a step. Step 702, which is optional, includes mapping each image of the X-ray stream, such as by converting each X-ray image to a 16-bit grayscale pixel map. Step 704 includes adjusting each X-ray image for subsequent processing, such as by adjusting the contrast of each X-ray image as needed. Step 704 can also include upscaling each image, such as via bicubic interpolation and further linearization, by way of non-limiting examples. Step 706 includes performing image segmentation on each X-ray image, which can include a sub-step 707a of performing edge detection on each X-ray image, and can include another sub-step 707b of performing object recognition within each X-ray image, such as by comparing image data in each X-ray image to a library of reference X-ray images stored in the computer memory. Step 708 includes object filtering the segmented X-ray images, which can be performed according to one or more various quality parameters. Step 710 includes fitting a shape, such as a circle, with respect to each reference feature 10 (e.g., hole) of the marker 8, which fitting can be performed according to LSQ techniques, such as an LSQ minimum error approximation. Step 712 includes further object filtering the X-ray images, which can be performed according to two (2) or more quality parameters. For example, one or more of the quality parameters in step 712 can involve calculations based on shape residuals. Step 712 can optionally be performed according to an iterative approach, in which the outcome of one or more of the filtering parameters is reapplied to each X-ray image, such as until a predetermined filtering standard or threshold is achieved for each X-ray image. The X-ray images processed according to step 712, particularly the marker 8 and its processed reference feature(s) 10 therein, are ready for co-registration (step 508) with the transform-processed images of the video stream.

Step 508 (co-registration of the video stream images and X-ray stream images) can include sub-steps 802, 804, 806, 808, and 810, which can each be referred to as a step. Step 802 includes performing nearest-neighbor interpolation on each set of paired video and X-ray images (hereinafter referred to as “image pairs”). Step 804 includes performing linear interpolation on each image pair. Step 806 includes performing B-spline interpolation on each image pair. Step 808 includes iteration of one or more of steps 802, 804, and 806, such as until a predetermined co-registration standard or threshold is achieved for each image pair. Step 810 includes performing object filtering on the image pairs, such as according to three (3) quality parameters. At step 510, the co-registered, filtered image pairs from step 508 are then combined by superimposing the X-ray images onto the paired video images in anatomically matching configurations to generate a continuous stream of AR images, which is then transmitted to the display at step 512.

The AR surgical imaging system 102 provides significant advantages over prior art surgical imaging systems, particularly with respect to surgical procedures that require precise targeting of implanted components with surgical instrumentation. Intramedullary (IM) nailing procedures are one non-limiting example of such procedures. In particular, even with fluoroscopy, aligning a drill bit to a distal locking hole of an IM nail can be difficult, especially if a surgeon is required to maneuver the drill while viewing the display of a fluoroscopic imaging device. The AR imagery of the embodiments described above allow the surgeon the ability to view, substantially in real-time, the relative position between the drill tip 205 and the distal locking holes of the IM nail. This can significantly reduce the amount of X-ray exposure to both the patient and the surgeon during an IM nailing procedure.

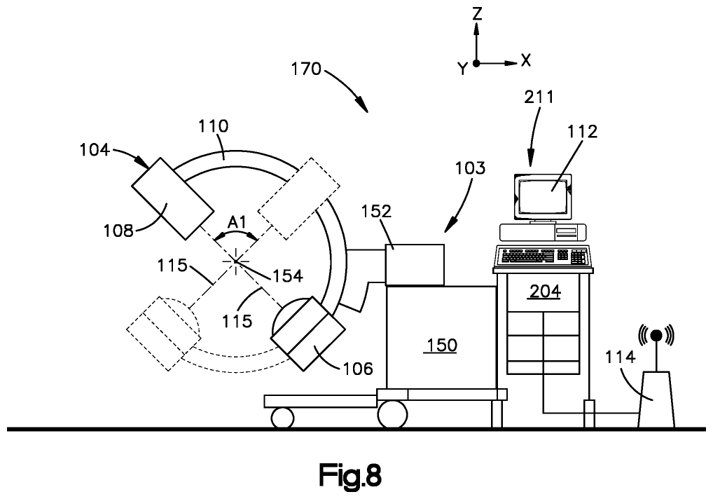

Referring now to FIG. 8, another example surgical imaging system 170 is shown for generating and displaying augmented imagery, such as a stream of augmented images for calculating the necessary orientation and length of anchors in three-dimensional (3D) space for anchoring an implant 12 to an anatomical structure 4. The imaging system 170 can include an imaging station 103 that includes a positioning mechanism, such as a robotic arm 110, that carries an imaging array that includes an imaging device 104, which is preferably a fluoroscopic imaging device 104. The surgical imaging system 170 of the present embodiment can be similar to the AR surgical imaging station 102 described above. For brevity and conciseness, the following description will focus on differences of system 170 with respect to system 102. Similar reference numbers will be used to denote similar components and subsystems of the systems 102, 170.

In the present embodiment, the fluoroscopic imaging device 104 is configured to obtain a first fluoroscopic image stream, taken at a first orientation at which beam axis 115 intersects pivot axis 154, and a second fluoroscopic image stream, taken at a second orientation (indicated by dashed lines) at which beam axis 115 intersects pivot axis 154. The first and second orientations are angularly offset from one another at a beam offset angle Al about the pivot axis 154. The beam offset angle Al can be in a range from about 10 degrees to about 170 degrees, and is preferably from about 60 degrees to about 120 degrees, and is more preferably from about 80 degrees to 100 degrees, and even more preferably is about 90 degrees. The fluoroscopic imaging device 104 is configured to transmit the first and second fluoroscopic image streams to the control unit 204 for image processing and augmentation.

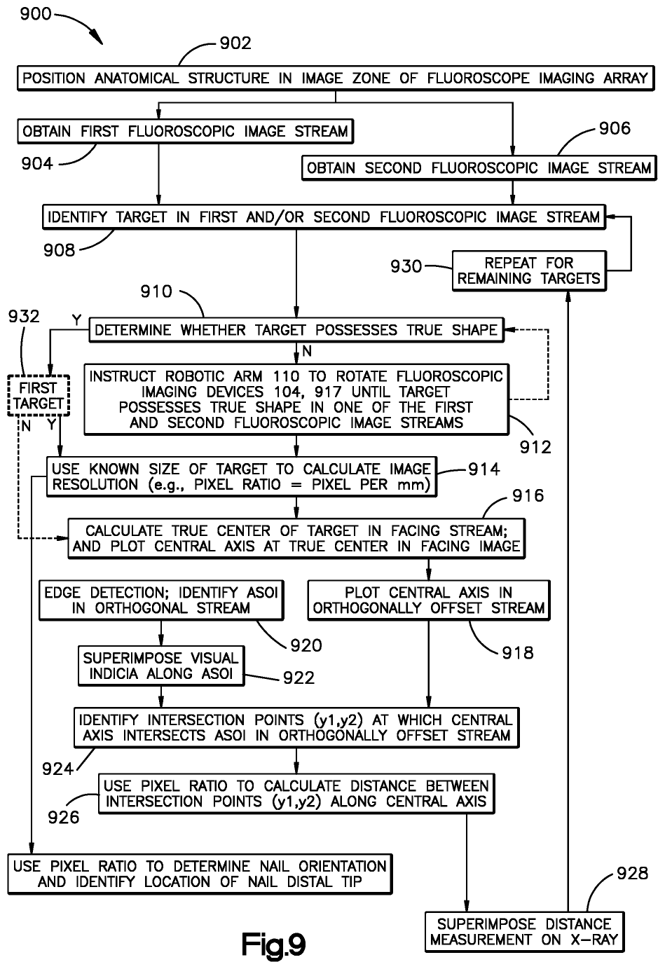

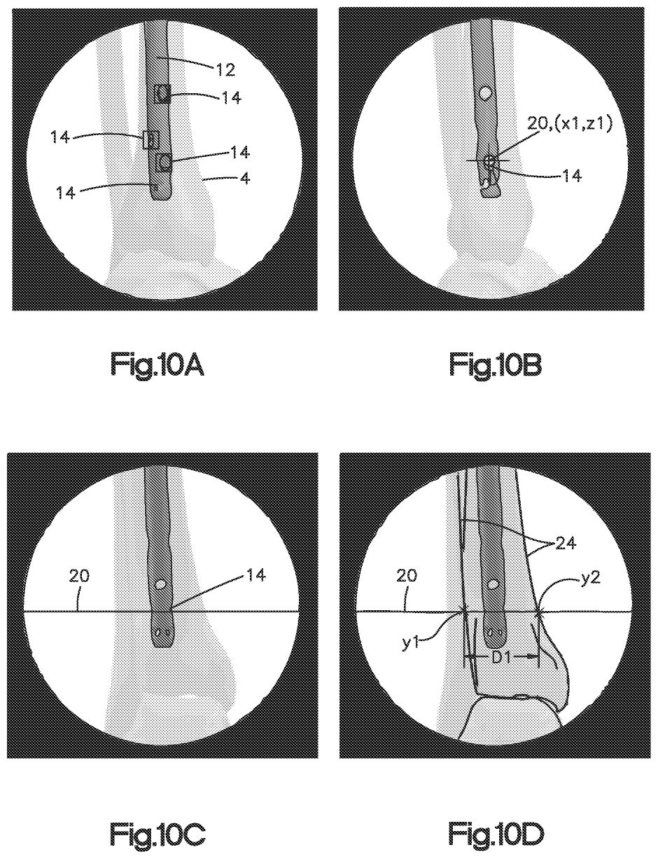

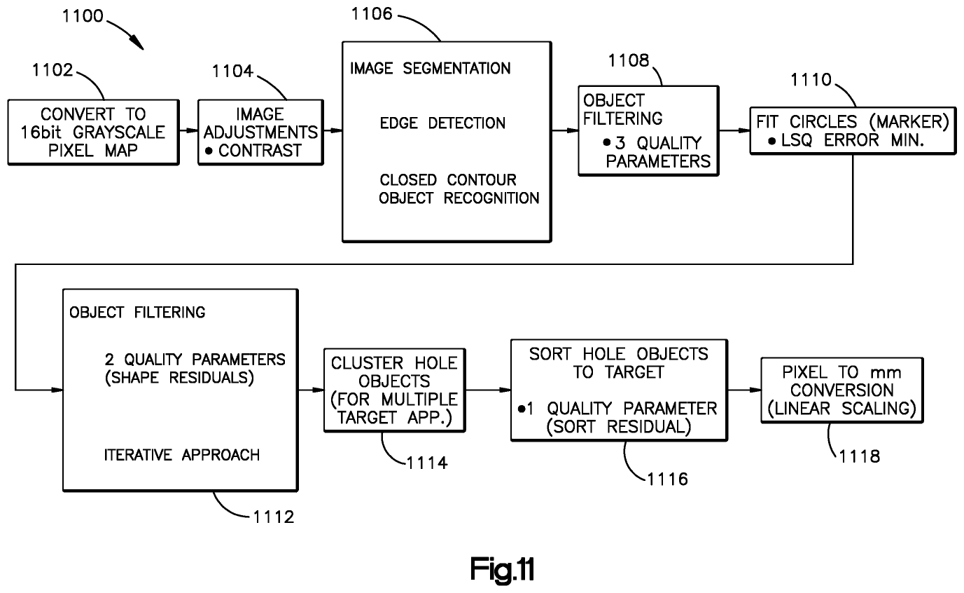

Referring now to FIG. 9, an example augmentation algorithm or process 900 will be described for calculating the necessary orientation and length of anchors in 3D space for anchoring an implant 12 to an anatomical structure 4. In relation to process 900, FIGS. 10A and 10B depict a first fluoroscopic image stream (generated at the first orientation) at various states of process 900, while FIGS. 10C and 10D depict the second fluoroscopic image stream (generated at the second orientation) at various states of process 900. Most, and optionally all, of the steps of process 900 are performed by the control unit 204 (e.g., by the processor 206 executing program instructions stored in the memory 214) on the first and second fluoroscopic image streams. The fluoroscopic images of the first and second fluoroscopic image streams can be delivered to the control unit 204 in DICOM format, by way of a non-limiting example. In the present embodiment, the control unit 204 and the AR display 112 are preferably incorporated into the computer station 211.

Process 900 can include steps 902, 904, 906, 908, 910, 912, 914, 916, 918, 920, 922, 924, 926, 928, 930, and 932. These steps can be categorized according to the following sub-routines of process 900: image resolution (steps 908, 910, 912, and 914); implant processing (steps 908, 910, 912, 914, 916, and 918); and anatomy processing (steps 920, 922, 924, and 926). It should be appreciated that some of the foregoing steps, can be applicable to multiple sub-routines. For example, process 900 utilizes target structures of the implant 12 for the image resolution sub-routine; thus steps 908, 910, 912, and 914 are utilized in both the image resolution and implant processing sub-routines. In other embodiments and processes, a separate reference marker 8 can be employed for the image resolution sub-routine, similar to the manner described above with reference to FIGS. 3B and 7 (see steps 700-712). Such as example process will be described in more detail below.

Step 902 includes positioning the anatomical structure 4 in the imaging zone 6 of the imaging array, particularly such that a ROI of the anatomical structure 4 can be intersected by the beam axis 115 at the first and second orientations. In the present example, the ROI includes an implant 12 residing within the anatomical structure 4. In the example illustrated embodiment, the implant 12 is an IM nail residing within the medullary canal of a tibia, and the ROI encompasses a distal portion of the IM nail, which includes a distal tip and anchoring structures, such as distal locking holes extending transversely through the IM nail at various orientations. It should be appreciated that the augmentation process 900 can be employed with other implant and anchor types.

Step 904 includes obtaining, by the control unit 204, a first fluoroscopic image stream of the ROI from the fluoroscopic imaging device 104 at a first orientation. Step 906 includes obtaining, by the control unit 204, a second fluoroscopic image stream of the ROI from the fluoroscopic imaging device 104 at a second orientation that is angularly offset from the first orientation at the offset angle A1. The first and second fluoroscopic image streams preferably show the implant 12, including one or more implant structures of interest (“ISOI”), which can also be referred to as implant “targets” 14. With particular reference to the IM nail shown in the illustrated embodiments, non-limiting examples of such targets thereof can include the distal tip, the distal locking holes 14, and an outer surface of the nail.

Step 908 includes processing the first and second fluoroscopic image streams, by the processor 206 executing instructions stored in the memory, to identify one or more targets 14 in one or both of the image streams (see FIG. 10A). For example, the processor 206 can convert each image of the streams into a pixel map, such as a 16-bit grayscale pixel map, and can additionally execute one or more cleaning algorithms, such as by adjusting the image contrast and/or applying one or more Gabor filters, such as from a circular Gabor filter bank. The processor 206 can also perform image segmentation and edge detection algorithms, such as Canny edge detector, by way of a non-limiting example, to identify edge patterns in one or both of the image streams and compare the edge patterns to a library of edge patterns associated with specific target 14 shapes stored in the memory. Step 908 can include executing an error calculation on the identified edge patterns and can identify or “register” the presence of a target 14 when the outcome of such error calculation falls within a threshold range.

Step 910 includes determining whether the target 14 possesses or presents its “true shape” in at least one of the image streams. As used herein, the term “true shape” means the shape of the target 14 when it directly faces the X-ray transmitter 106, or, stated differently, when viewed along a beam axis 115 that orthogonally intersects the reference plane of the target 14. To determine whether the target 14 presents its true shape in the image stream, the control unit 204 can process the target 14 to calculate a deviation between its true shape, as logged in the library in the memory, and the shape presented in the respective image stream. For example, with reference to the illustrated embodiment shown in FIG. 10A, in which the target 14 is a locking hole, the processor 206 can identify whether the viewed shape is elliptical and thus deviates from a circle true shape. In step 912, if the deviation result exceeds a minimum threshold, the control unit 204 instructs the robotic arm 110 to rotate the imaging array about axis 154 until one of the fluoroscopic imaging devices 104, 109 obtains a view of the true shape of the target 14 (i.e., when the associated X-ray transmitter 106 directly faces the target 14), as shown in FIG. 10B. Accordingly, this fluoroscopic imaging stream can be referred to hereafter as the “facing stream”, and the other fluoroscopic imaging stream can be referred to as the orthogonally offset stream. It should be appreciated that steps 910 and 912 can optionally be repeated as needed to provide a confirmation mechanism for step 910.

After the true shape of the target 14 has been confirmed, step 914 can be performed, which includes using a known size of the target 14 (as retrieved from the library), such as a width thereof, to calculate an image resolution of the facing stream, which can also approximate with a high degree of certainty the image resolution of the orthogonally offset stream. For example, when the target 14 has a true shape that is a circle, such as when the target 14 is a hole, such as a locking hole, the known size can be the diameter of the circle. The processor 206 can calculate the image resolution by counting the number of pixels along a line extending along the diameter of the circle, and can output the image resolution as a pixel ratio, particularly the quantity of pixels per unit distance, such as pixels per mm, for example. It should be appreciated that the calculated pixel ratio, in combination with image processing of the implant 12, can also assist with determining various parameters of the implant, such as a longitudinal axis of the implant 12, the implant's rotational orientation about the longitudinal axis, and the location of the distal-most end or “tip” of the implant 12, by way of non-limiting examples.

Step 916 includes using the calculated image resolution to identify the center of the target 14 in the associated image stream. This step includes plotting a central axis 20 of the target 14 in the facing stream, as shown in FIG. 10B. For example, plotting the central axis 20 in the facing image stream can include registering a center point of the target 14, such as by registering a coordinate for the center point, such as coordinates (x1, z1) along respective x- and z-axes in a cartesian coordinate system. This step can also include superimposing visual indicia, such as highlighted pixilation, in the facing stream at the center coordinates (x1, z1) in a manner providing a visual reference of the location of the central axis 20. Step 918 includes plotting the central axis 20 in the orthogonally offset image stream (FIG. 10C), which can include registering a straight line along the x1 coordinate in the orthogonally offset stream. The orthogonally offset stream can be defined with respect to x- and y-axes in the cartesian coordinate system.

Step 920 includes identifying one or more anatomical structures of interest (“ASOI”) in one or both of the image streams. This step can be performed by the processor 206 executing edge detection and/or cleaning algorithms (such as those described above at step 908) to thereby identify edge geometries and patterns indicative of specific portions of the anatomical structure 4, such as the outer cortical surfaces of a bone, such as a longbone, such as a tibia in which the IM nail is implanted, by way of a non-limiting example. Step 922 includes augmenting one or both of the facing and orthogonally offset image streams, such as by superimposing visual indicia onto the image streams. For example, as shown in FIG. 10D, reference lines and/or contours 24 (e.g., via highlighted pixilation) can be superimposed onto edges of the outer cortical surface of one or more bones in the image stream. This can be performed according to a Circular Hough transform, by way of a non-limiting example. Such visual indicia can provide assistance during step 924, which includes identifying intersection points at which the central axis 20 intersects ASOI in the orthogonally offset image stream. In the illustrated example, the ASOI is the outer cortical surface of the longbone in which the IM nail resides. The intersection points (y1, y2) can be plotted with respect to the y-axis of the cartesian coordinate system, for example. The intersection points (y1, y2) can be plotted by a user, such as a surgeon or medical technician, with the assistance of the user interface 216 and controls 219, such as via mouse click or contacting a stylus against the AR display 112 surface (if touch-screen capable), by way of non-limiting examples. In other embodiments, the intersection points (y1, y2) can be plotted autonomously by the processor 206. In such embodiments, the processor 206 can generate visual indicia at the autonomously selected intersection points (y1, y2), and can additionally provide the user with an option of accepting these intersection points (y1, y2) or inputting alternative intersection points directly via the controls 219.

Step 926 includes using the image resolution (e.g., pixel ratio) to calculate a distance D1 along the central axis between the intersection points (y1, y2). With reference to the illustrated example, this axial distance D1 can be used to select a locking screw having sufficient length to extend through the target 14 locking hole and purchase within the near and far cortex of the bone. Step 928 includes superimposing the distance D1 measurement alongside the associated axis on an X-ray image, such as an image of the orthogonally offset stream. Step 930 includes repeating various steps of process 900 for the remaining targets 14 of the implant 12, such as steps 908, 910, 912, 914, 916, 918, 920, 922, 924, 926, 928, for example. Process 900 can optionally provide a bypass feature, such as step 932, which can bypass step 914 for subsequent targets 14. The output of process 900 can be a reference X-ray image that identifies each target 14, and depicts the superimposed axis 20 thereof and the associated distance D1 measurement for each target 14.

Referring now to FIG. 11, an example of an image resolution sub-routine 1100 that employs a separate, ex vivo reference marker 8 will be described. Sub-routine 1100, which can also be referred to as a process or method, can include steps 1102, 1104, 1106, 1108, 1110, and 1112, which are performed by the control unit 204 (e.g., by the processor 206 executing program instructions stored in the memory 214) on the X-ray images received from the first and second fluoroscopic imaging devices 104, 109. These X-ray images can be transmitted to the control unit 204 in DICOM format, by way of a non-limiting example. Step 1102, which is optional, includes mapping each image of at least one of the first and second X-ray streams, such as by converting each X-ray image thereof to a 16-bit grayscale pixel map, which can have various pixel matrix configurations. By way of non-limiting examples, the pixel matrix configuration of the pixel map can be from 8×32 to 16×32 and preferably from 8×64 to 16×64, depending on the C-arm employed. As another non-limiting example, the pixel map can be an 8-bit pixel map. Step 1104 includes adjusting each X-ray image for subsequent processing, such as by adjusting the contrast of each X-ray image as needed. Step 1104 can also include upscaling each image, such as via bicubic interpolation and further linearization, by way of non-limiting examples.

Step 1106 includes performing image segmentation on each X-ray image, which can include a sub-step of performing edge detection on each X-ray image, and can include another sub-step of performing object recognition within each X-ray image, such as by comparing image data in each X-ray image to a library of reference X-ray images stored in the computer memory 214. Step 1108 includes object filtering the segmented X-ray images, which can be performed according to one or more various quality parameters. Step 1110 includes fitting a shape, such as a circle, with respect to each reference feature (e.g., hole) of the marker 8, which fitting can be performed according to LSQ techniques, such as an LSQ minimum error approximation. Step 1112 includes further object filtering the X-ray images, which can be performed according to two (2) or more quality parameters, which can be different quality parameters than those of step 1108. For example, one or more of the quality parameters in step 1112 can involve calculations based on shape residuals. Step 1112 can optionally be performed according to an iterative approach, in which the outcome of one or more of the filtering parameters is reapplied to each X-ray image, such as until a predetermined filtering standard or threshold is achieved for each X-ray image.

Steps 1114 and 1116 can be included in process 1100 when each X-ray image depicts multiple markers 8, each having a plurality of reference features, or when each X-ray image depicts multiple groupings or “clusters” of reference features. Step 1114 includes clustering the reference features. Step 1116 includes sorting the reference features according their associated marker 8 or associated region of the X-ray image, which sorting can be performed according to at least one (1) quality parameter, which can involve a sort residual calculation. Step 1118 includes calculating a pixel ratio for each X-ray image (e.g., pixels per mm), which can be performed according to a linear scaling function.

It should be appreciated that the processes, steps, and techniques described above with reference to FIGS. 8-11 can be adapted as needed to calculate the required orientation and length of anchors for affixing other types of implants to adjacent anatomy. Such implants can include bone plates, bone screws, intervertebral cages, and the like.

While example embodiments of devices for executing the disclosed techniques are described herein, the underlying concepts can be applied to any control unit, computing device, processor, or system capable of communicating and presenting information as described herein. The various techniques described herein can be implemented in connection with hardware or software or, where appropriate, with a combination of both. Thus, the methods and apparatuses described herein can be implemented, or certain aspects or portions thereof, can take the form of program code (i.e., instructions) embodied in tangible non-transitory storage media, such as floppy diskettes, CD-ROMs, hard drives, or any other machine-readable storage medium (computer-readable storage medium), wherein, when the program code is loaded into and executed by a machine, such as a computer, the machine becomes an apparatus for performing the techniques described herein. In the case of program code execution on programmable computers, the computing device will generally include a processor, a storage medium readable by the processor (including volatile and non-volatile memory and/or storage elements), at least one input device, and at least one output device, for instance a display. The display can be configured to display visual information. For instance, the displayed visual information can include fluoroscopic data such as X-ray images, fluoroscopic images, orientation screens, or computer-generated visual representations.

The program(s) can be implemented in assembly or machine language, if desired. The language can be a compiled or interpreted language, and combined with hardware implementations.

The techniques described herein also can be practiced via communications embodied in the form of program code that is transmitted over some transmission medium, such as over electrical wiring or cabling, through fiber optics, or via any other form of transmission. When implemented on a general-purpose processor, the program code combines with the processor to provide a unique apparatus that operates to invoke the functionality described herein. Additionally, any storage techniques used in connection with the techniques described herein can invariably be a combination of hardware and software.

While the techniques described herein can be implemented and have been described in connection with the various embodiments of the various figures, it is to be understood that other similar embodiments can be used or modifications and additions can be made to the described embodiments without deviating therefrom. For example, it should be appreciated that the steps disclosed above can be performed in the order set forth above, or in any other order as desired. Further, one skilled in the art will recognize that the techniques described in the present application may apply to any environment, whether wired or wireless, and may be applied to any number of such devices connected via a communications network and interacting across the network. Therefore, the techniques described herein should not be limited to any single embodiment, but rather should be construed in breadth and scope in accordance with the appended claims.

What is claimed:

1. A medical imaging system, comprising:
a robotic arm carrying a fluoroscopic imaging device having an x-ray emitter, wherein the fluoroscopic imaging device is configured to generate fluoroscopic image data of an anatomical structure along a beam axis, the robotic arm being manipulatable for adjusting a relative position between the fluoroscopic imaging device and the anatomical structure;
a video imaging device configured to generate video image data of the anatomical structure along a camera sightline axis;
a marker positioned with respect to the anatomical structure, the marker defining at least one reference feature configured to be captured in the fluoroscopic image data and the video image data;
a processor in communication with the fluoroscopic imaging device and the video imaging device, the processor further in communication with a memory having instructions stored therein, wherein the processor is configured to execute the instructions upon the fluoroscopic image data and the video image data and responsively:
register a reference position of the at least one reference feature relative to the anatomical structure in the fluoroscopic image data and the video image data; and
generate an augmented image stream that shows one of the fluoroscopic image data and the video image data overlaid onto the other of the fluoroscopic image data and the video image data such that the reference positions are co-registered; and
a display in communication with the processor, wherein the display is configured to present the augmented image stream of the anatomical structure substantially in real time.

2. The medical imaging system of claim 1, wherein the marker is positioned adjacent the anatomical structure at an ex vivo location.

3. The medical imaging system of claim 2, wherein:
the fluoroscopic image data comprises a fluoroscopy stream showing the anatomical structure and the reference marker,
the video image data comprises a video stream showing the anatomical structure and the reference marker, and
the augmented image stream comprises an adjusted version of the video stream superimposed with an adjusted version of the fluoroscopy stream such that the reference marker occupies the same area in the superimposed adjusted versions of the video and fluoroscopy streams.

4. The medical imaging system of claim 3, wherein the video imaging device is attached adjacent the x-ray emitter.

5. The medical imaging system of claim 4, wherein the camera sightline axis is substantially parallel to the beam axis.

6. The medical imaging system of claim 3, further comprising an instrument having a distal tip configured to operate upon the anatomical structure, wherein the augmented image shows a substantially live stream of the distal tip positioned with respect to the anatomical structure when the distal tip is within a field of view of the video stream.

7. The medical imaging system of claim 6, wherein the instrument has a handle portion, the distal tip extends from the handle portion along an instrument axis, and the video imaging device is a camera attached to the instrument such that that the camera sightline axis is substantially parallel to the instrument axis.

8. The medical imaging system of claim 7, wherein the instrument is a drill, the distal tip is defined by a drill bit coupled to the drill, and the adjusted version of the fluoroscopy stream in the augmented image shows an implant inserted within the anatomical structure.

9. A method, comprising:
generating a fluoroscopic stream of images of an anatomical structure;
generating a video stream of images of the anatomical structure;
co-registering the fluoroscopic stream of images with the video stream of images; and
depicting, on a display, an augmented image stream that includes the co-registered fluoroscopic stream of images overlaid over the co-registered video stream of images.

10. The method of claim 9, wherein the fluoroscopic stream of images depicts an implant residing in the anatomical structure.

11. The method of claim 10, further comprising manually manipulating a surgical instrument toward the anatomical structure, such that at least a distal tip of the surgical instrument is depicted in at least one of the fluoroscopic and video stream of images.

12. The method of claim 11, wherein the distal tip is radiopaque, and the distal tip is depicted in both of the fluoroscopic and video stream of images.

13. The method of claim 12, wherein the implant is an intramedullary nail having at least one distal locking hole, and the surgical instrument is a power drill.

14. A surgical system, comprising:
a robotic arm carrying a fluoroscopic imaging device having an x-ray emitter, wherein the fluoroscopic imaging device is configured to generate a first stream of fluoroscopic images of an anatomical structure along a first beam axis at a first orientation relative to the anatomical structure, and the fluoroscopic imaging device is also configured to generate a second stream of fluoroscopic images of the anatomical structure along a second beam axis at a second orientation relative to the anatomical structure, wherein the second beam axis intersects the first beam axis and is substantially perpendicular to the first beam axis, and the robotic arm is manipulatable for adjusting a relative position between the fluoroscopic imaging device and the anatomical structure;
a processor in communication with the fluoroscopic imaging device and the robotic arm, the processor further in communication with a memory having instructions stored therein, wherein the processor is configured to execute the instructions upon the first and second streams of fluoroscopic images and responsively:
identify at least one anchor hole in an implant that resides within the anatomical structure;
reposition the fluoroscopic imaging device so that the first beam axis extends orthogonal to the at least one anchor hole; and
plot, in the second stream of fluoroscopic images, a reference axis that extends centrally through the at least one hole; and
a display in communication with the processor, wherein the display is configured to depict an augmented version of the second stream of fluoroscopic images that shows the reference axis overlaying the anatomical structure.

15. The medical imaging system of claim 14, further comprising a user interface having inputs in communication with the processor, wherein the inputs are configured to allow a user to select locations along the reference axis, and the processor is configured to responsively calculate a distance along the reference axis between the selected locations.

16. The medical imaging system of claim 15, wherein the processor is further configured to execute additional instructions upon at least one of the first and second streams of fluoroscopic images data and responsively display visual indicia within the augmented version of the second stream along an anatomical landmark of the anatomical structure.

17. The medical imaging system of claim 16, wherein the processor is further configured to execute further additional instructions upon at least one of the first and second streams of fluoroscopic images data and responsively generate an augmented version of the first stream of fluoroscopic images that shows additional visual indicia along the anatomical landmark.

18. The medical imaging system of claim 17, wherein the inputs are configured to allow a user to toggle between the augmented version of the first stream and the augmented version of the second stream.

19. A method, comprising:
generating a first fluoroscopic stream of images along a first beam axis, the first fluoroscopic stream of images showing an implant residing in an anatomical structure;
generating a second fluoroscopic stream of images of the anatomical structure along a second beam axis that intersects the first beam axis at an angle;
processing the first and second fluoroscopic streams of images with a processor in communication with memory, the processing step comprising:
identifying a reference feature of the implant;
calculating a pixel ratio of the reference feature in pixels per unit length;
adjusting an orientation of the first beam axis, thereby causing the first beam axis to extend orthogonal to the reference feature;
generating a reference axis extending centrally through the reference feature such that the reference axis is parallel with the first beam axis; and
depicting the second image stream on a display, wherein reference axis is depicted in the second image stream overlaying the anatomical structure.

20. The method of claim 19, further comprising calculating a distance along the reference axis between two reference points of the anatomical structure.

21. The method of claim 20, wherein the two reference points are selected by a user.
